(19) United States
(12) Patent Application Publication
(10) Pub. No.: US 2006/0111552 A1
(43) Pub. Date: May 25, 2006

(54) Metal binding precursors for the synthesis of peptide-metal conjugates

(76) Inventors: Philip John Blower, Canterbury (GB); William Edward Greenland, Amersham (GB)

Correspondence Address: FOX ROTHSCHILD O'BRIEN & FRANKEL LLP;PRINCETON PIKE CORPORATE CENTER, 997 LENOX DRIVE, BUILDING 3, LAWRENCEVILLE, NJ, 08648, US

(21) Appl. No.: 10/526,718

(22) PCT Filed: Sep. 4, 2003
(86) PCT No.: PCT/GB03/03841

§ 371 Date: Oct. 3, 2005

(30) Foreign Application Priority Data
Sep. 5, 2002 (GB) 0220619.1

(51) Int. Cl. C07K 14/585; A61K 38/23; C07F 5/00

(52) U.S. Cl. 530/307; 530/400; 534/11

(57) ABSTRACT

Specific metal-chelating precursors incorporating a pendant protected (e.g. with Fmoc) amino acid functionality are synthesised. The pendant amino acid functionality allows the chelator to be inserted into a synthetic peptide sequence during standard solid-phase peptide synthesis at any predetermined position in the sequence, in place of lysine or any other amino acid, or in addition to native amino acids. An example is a conjugate incorporating Fmoc-protected L-lysine and the technetium-binding group hynic (hydrazinonicotinamide), shown as molecule 1 in FIG. 1 of the accompanying drawing. These molecules permit synthetic approaches with greater flexibility and control of the site of labelling than conventional methods. They are particularly suited to development of combinatorial libraries of radiolabelled peptides, which will be especially significant in the development of radiopharmaceuticals targeted towards the many new cancer-related targets likely to be identified in the near future through developments in proteomics.

This invention relates to precursors for use in the development and synthesis of radiolabelled biological molecules, in particular synthetic peptides, in which the radiolabel is a metallic radionuclide. The radiolabelled molecules are for use in medical diagnosis by single photon emission imaging or positron emission tomographic imaging, or in targeted radionuclide therapy.

### BACKGROUND TO THE INVENTION

Direct imaging of a variety of biochemical processes is now practicable through the development of radiopharmaceuticals targeted towards specific disease-associated molecular targets. This was made possible by the new discoveries in the field of disease-related changes in cellular communication and metabolism, especially in cancer. To support these new diagnostic applications, methods for linking radioisotopes to the appropriate targeting biomolecules were required to replace the simple metal chelates and ions used previously. In the 1980s and 1990s methods were developed for labelling biomolecules, especially monoclonal antibodies, with radionuclides such as technetium-99m and indium-111. In its most developed form, this typically entailed covalent attachment of a bifunctional chelator to a protein, followed by labelling with the radiometal, or even synthesis of a radiometal bifunctional chelate which was subsequently attached to the protein. As recognition grew in the 1980s that monoclonal antibodies are too large to offer ideal pharmacokinetics, focus shifted onto smaller molecules such as antibody fragments and especially smaller peptides targeted towards receptors present in lesions such as tumours and thrombi. Radiolabelled octreotide, a small peptide that binds to somatostatin receptors expressed in many cancers, is the leading example but several others (e.g. vasoactive intestinal peptide, bombesin etc.) are now finding widespread clinical use.

Although the transition to smaller molecules brought with it the opportunity to use peptides produced by solid phase peptide synthesis (SPPS) rather than proteins of biological origin, the same methods were used to label them as had been used to label antibodies. These methods have several disadvantages, which are more problematic with small peptides than with large proteins. The most suitable sites for attachment of a bifunctional chelator in most peptides are the ε-amino groups of lysine residues and the N-terminus, because they are very reactive nucleophiles and form very unreactive covalent links with the chelator. If there is more than one lysine, the site of modification becomes uncertain. For instance, if the peptide has two lysines, together with the N-terminus these will present three possible sites for conjugation, hence forming as many as eight products when treated with an active-ester-containing bifunctional chelator or radiolabelled bifunctional chelate. Each of these products will have a different biodistribution and different affinities for the target (some of them may have lost all target affinity) and such a mixture is not acceptable for clinical use. Moreover, one or more of the lysines may be essential to the biological activity of the peptide. A simple solution has been to incorporate the chelator, or a radiolabelled chelate or organic prosthetic group, as the last step of SPPS. This, however, has the limitation that the chelator has to be at one end of the peptide chain, which is frequently essential to the biological activity of the peptide.

The state of the art in linking radiometals to peptides encompasses a number of approaches. Some have the advantage of incorporating the metal binding sequence during SPPS, and others have the advantage of incorporating chelators that are specifically designed for the particular metal. None, however, have both of these advantages. For example, technetium-chelating amino acid sequences such as gly-gly-cys are incorporated during SPPS or recombinant protein production, but this sequence is not ideal for its purpose, and merely represents the best that can be achieved for chelating the TcO³⁺ core using “standard” amino acids (i.e. those coded through tRNAs). Likewise, polyhistidine sequences such as hexahistidine can be incorporated during SPPS, but again they merely represent the best sequence of coded amino acids achievable for chelating the Tc(CO)₃⁺ core. Conversely, the synthetic technetium ligand hynic (hydrazinonicotinamide) probably represents the most convenient and efficient labelling system to date for use with Tc-99m, but it has so far only been used by conjugating it to a pre-formed peptide, with all the associated problems outlined above. An alternative that offers convenience of labelling is the “direct labelling” method in which antibodies and peptides containing disulfide bonds can be reduced and labelled with Tc-99m or Re-188. However, the chemistry of these methods is poorly understood, and there are major stability and biological activity problems as demonstrated by the work of several groups world wide with antibodies and somatostatin analogues.

There is therefore a need for a more versatile and controlled approach to the synthesis of peptide conjugates for radiolabelling.

### SUMMARY OF THE INVENTION

According to the present invention, metal-chelating precursors, designed to bind specific metallic radionuclides and incorporating a pendant protected (e.g. Fmoc) amino acid functionality, are synthesised. This allows an advance on conventional synthesis of peptide-chelator conjugates in that the chelator is attached to an amino acid before rather than after SPPS assembly of the peptide chain, with the advantages identified below.

According to the present invention there is provided a chelator-derivatised amino acid comprising: 1) an optionally protected primary or secondary amino group; 2) a carboxylic acid group; 3) a chelator group capable of binding a metallic radionuclide.

The chelator may comprise any group capable of chelating or binding a metal. Preferably, the chelator is capable of the specific, kinetically stable attachment of a metallic radionuclide chosen for diagnostic imaging or treatment. Preferably, the chelator is capable of chelating or binding at least 95% of the radionuclide within 1 hour under typical radiolabelling conditions (for example, at a chelator concentration of 1 μM and a ratio of chelator to radionuclide of 100:1, at 25° C.) and the conjugate is stable for at least 5 minutes, preferably 2 days, in blood serum under physiological conditions.

Preferably the metal binding functional group will be specifically designed to form a kinetically stable complex with a suitable metallic gamma- or positron-emitting radionuclide for diagnostic imaging (including but not limited to technetium-99m, indium-111, copper radioisotopes, and lanthanide radioisotopes) or a particle-emitting metallic radionuclide for radionuclide therapy (including but not limited to rhenium-186, rhenium-188, copper-64, copper-67, and lanthanide radioisotopes). Suitable chelating or metal binding groups may preferably be chosen from several structures including but not limited to the hydrazinonicotinamide group, di- or poly-thiol groups, macrocyclic ligands incorporating amine, thioether, or phosphine donor groups, or polyaminocarboxylate groups.

Preferably, the chelator group (metal binding group) is a hynic (hydrazinonicotinamide) group. The hydrazine group of hynic may be protected. Preferred protecting groups for the hydrazine group of hynic include Boc or trifluoroacetyl protecting groups.

In one aspect of the invention, the hydrazine group is protected by a suitable protecting group such as Boc (as illustrated as molecule 1 in FIG. 1 of the accompanying drawings) for the purposes of solid phase peptide synthesis, or trifluoroacetyl (as in molecule 2 in FIG. 2) for the purposes of preventing cleavage or alkylation or acylation of the N—N bond during subsequent manipulation of the peptide.

The amino acid may comprise any compound comprising a primary or secondary amino group and a carboxylic acid group. Preferably, the amino acid is an a-amino acid. More preferably, the amino acid is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. More preferably, the amino acid is lysine or a homologue thereof, such as ornithine. Homologues of lysine comprise amino acids in which the C₄ alkylene chain of lysine is replaced by a C₁₋₆ alkylene chain, such as by a C₃ chain in ornithine. More preferably, the amino acid is selected from lysine and ornithine. More preferably, the amino acid is lysine.

Preferably, the amino acid is an L-amino acid.

The amino acid functionality may preferably but not necessarily be derived from an enantiomerically pure amino acid with an amine-containing side chain, such as L-lysine or L-ornithine or a similar homologue.

In one aspect of the invention, the amine and carboxylic acid groups are embodied in the amino acid L-lysine (as in molecule 1 in FIG. 1 of the accompanying drawings) or its homologues including but not limited to L-ornithine.

Preferably, the amino acid is L-lysine or L-ornithine, more preferably L-lysine.

The amino group of the amino acid may be protected. Preferred amino protecting groups include an Fmoc protecting group.

Preferably, the chelator is bound covalently to the amino acid. Preferably, the chelator is bound to the amino acid via a functional group on a side chain of the amino acid, for example via the amino functional group on the side chain of lysine or ornithine.

In one aspect, the present invention provides a chelator-derivatised amino acid into which three functionalities are incorporated: 1) a primary or secondary amino group, which may be protected for the purposes of solid phase peptide synthesis using standard protecting groups including but not limited to Fmoc; 2) a carboxylic acid group; 3) a chelating or metal-binding group designed for the specific, kinetically stable attachment of the metallic radionuclide chosen for diagnostic imaging or treatment.

The invention further provides a method of synthesising a peptide comprising a chelator, the method comprising incorporation into the peptide of a chelator-derivatised amino acid according to the present invention. Typically, the peptide will be synthesised incorporating the chelator-derivatised amino acid prior to chelation of a radionuclide. However, the present invention further provides chelator-derivatised amino acids to which radionuclides are chelated and their use in the synthesis of radiolabelled peptides.

The peptide may be synthesized by solid phase peptide synthesis.

In one aspect the invention provides a method of synthesising a peptide modified with a chelator according to the present invention, in which the chelator is incorporated at any pre-determined position in the sequence during conventional solid phase peptide synthesis, by using chelator-derivatised amino acids according to the present invention as the amino acid building blocks.

The invention further provides a peptide obtained according to a method of the present invention. The invention further provides a peptide obtained according to a method of the present invention wherein the peptide is radiolabelled and use thereof in therapy and diagnosis.

The invention further provides a peptide comprising the amino acid sequence of salmon calcitonin in which lysine-18 is replaced by lysine-hynic. In one aspect, the invention further provides a derivative of salmon calcitonin (molecule 3) in which lysine-18 is replaced by a hynic derivative as shown in FIG. 3, and which is thus suitable, after the necessary deprotection, for convenient radiolabelling with technetium isotopes for purposes of diagnostic imaging of disease sites in which receptors are abundant. Calcitonin receptors are particularly abundant in osteoclasts and have been detected in various tumours and tumour-derived cells including human ovarian small cell carcinoma, breast cancer, prostate cancer and neuroblastoma as well as bone tumours (osteoclastoma) and bone cancers and metastases with increased osteoclast activity.

The chelating amino acids of the present invention are used in protected form as amino acid building blocks and can be inserted into a synthetic peptide sequence during SPPS at any predetermined position in the sequence, in place of lysine or any other amino acid, or in addition to native amino acids. These advantages overcome the problems associated with conventional methods described above. They are particularly suited to development of combinatorial libraries of radiolabelled peptides, which will be especially significant in the development of peptide radiopharmaceuticals targeted towards the many new cancer-related targets likely to be identified in the near future through developments in proteomics. Thus, the present invention further provides a library of peptides obtained according to a method of the present invention. The present invention further provides a library of peptides obtained according to a method of the present invention wherein the peptide is radiolabelled.

It has been found that when a histidine located next in the sequence to the chelator-derivatised amino acid such as hynic-lysine, the histidine plays a cooperative role in binding the metal and affords advantages for stability of the conjugate not seen in other chelator-derivatised molecules. It is also believed that having two chelator-derivatised amino acids next to each other in the sequence has advantages for metal binding. For example, two successive amino acids with bidentate chelators will provide a tetradentate donor set.

The radiolabelled peptides and libraries thereof may be used in the discovery of effect molecules such as peptide-based radiopharmaceuticals targeted towards therapeutic targets, discovery of novel biologics, and the discovery and characterization of peptide folds (determined by the presence of a metal linkage) allowing the rational design of novel biologics.

The advantages of the chelating amino acid method over conventional post-SPPS conjugation may be summarised as follows:
1. The method is versatile and flexible: it incorporates the chelator site-specifically anywhere in sequence, not just terminally or at one or more lysines.
2. It can leave lysines unmodified.
3. It is economic since no post-SPPS modification is needed.
4. It is suitable for a combinatorial approach to generate libraries of peptide radiopharmaceuticals from which imaging agents for specific targets can be selected.

In addition, the proposed chelating amino acid building blocks are advantageous over those in the literature in that they are suitable for use with metals useful in nuclear medicine whereas those in the literature are not.

The advantages of the chelating amino acid method over conventional chelating amino acid sequences (such as gly-gly-cys or poly-his, which are at present the only chelating structures that are incorporated during SPPS) may be summarised as follows:
1. It is versatile and flexible, in that it can be used for any metal since a specifically designed synthetic chelator can be used, and there is no need to rely on coded amino acids which are not ideal chelators.
2. It gives total control over the labelling site.

### BRIEF DESCRIPTION OF THE FIGURES

FIG. 1 shows a scheme describing the synthesis of such a chelating amino acid building block together with its structure (molecule 1). Molecule 1 incorporates an amino group in Fmoc protected form (labelled 1), a carboxylic acid group (labelled 2) and the technetium-binding group hynic in Boc protected form (labelled 3).

FIG. 2 shows, by way of example, the structure of a synthetic peptide (molecule 2), prepared using molecule 1, in which the hydrazide group is protected by a trifluoroacetyl group.

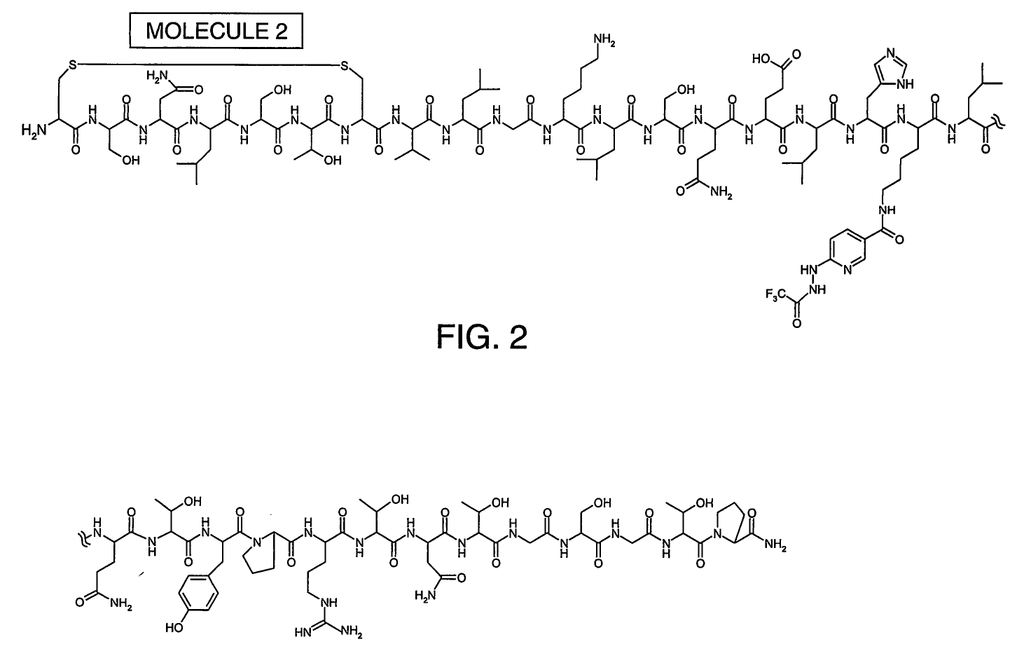

FIG. 3 shows, by way of example, the probable structure insofar as can be established by the available spectroscopic data and by comparison with the literature, of a radiolabelled derivative of molecule 2 (molecule 3).

Specific embodiments of the invention will now be described by way of examples.

### EXAMPLES

### Example 1

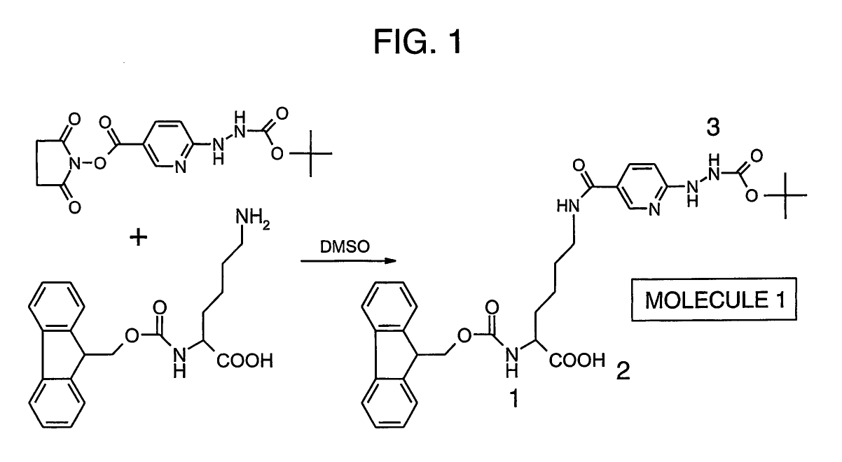

Synthesis of Fmoc-lysine-hynic-Boc (molecule 1, see FIG. 1). The synthetic route is summarised schematically in FIG. 1. To the N-hydroxysuccinimide ester of hynic-Boc (1.79 mmol), prepared by a literature method, in ˜10 mL dimethylsulfoxide is added 220 mg (0.597 mmol) of N-α-Fmoc-Lys (purchased commercially). An additional 5 mL of dimethylsulfoxide is added to aid solubility and the mixture is stirred overnight at room temperature. The mixture becomes a clear yellow solution after 60 minutes, then becomes cloudy after 180 minutes. This reaction mixture is added slowly to 60 mL of H₂O with stirring, after which the pH is 4. The mixture is stirred overnight at room temperature. The crude precipitated product is filtered off and washed with 5 mL H₂O and dried under vacuum to give 460 mg of an off-white solid. The crude product is dissolved in methanol and purified by normal phase HPLC using isocratic elution with methanol. The broad second peak is collected and dried under vacuum. The product is an off white-yellow solid, 229 mg, 64% yield relative to N-α-Fmoc-Lys. Analysis by reversed phase HPLC (RP-HPLC) and electrospray mass spectrometry (ES-MS) gives a major peak at 26.91 minutes with m/z (relative abundance) shown in the table below:

| | Observed | Calculated |
|---|---|---|
| Charged species assigned | m/z | m/z |
| [N-α-Fmoc-N-ε-(Hynic-Boc)-Lys+H]⁺ | 604.1 (100) | 604 (100) |
| [(N-α-Fmoc-N-ε-(Hynic-Boc)-Lys)₂+H]⁺ | 1207.1 (16) | 1207 (100) |
| [N-α-Fmoc-N-ε-(Hynic-CO₂H)-Lys+H]⁺ | 548.1 (53) | 548 (100) |
| [N-α-Fmoc-N-ε-(Hynic)-Lys+H]⁺ | 504.3 (30) | 504 (100) |

NMR and IR spectra and elemental analysis are consistent with the proposed product.

### Example 2

Synthesis of trifluoroacetyl-protected hynic-modified salmon calcitonin (molecule 2). The peptide is synthesised using standard Fmoc SPPS with the following modifications. N-α-Fmoc-N-ε-(Hynic-Boc)-Lys (molecule 1) is used in place of N-α-Fmoc-Lys at position 18 of native salmon calcitonin. All amino aids are used at ×10 excess except for molecule 1, which is used at ×5 molar excess (30.2 mg). The completed 32-amino acid sequence is cleaved from the resin by treatment with trifluoroacetic acid for 3 hours. After the precipitation with diethyl ether the product is reconstituted with 2.5 mL H₂O. Analytical RP HPLC ES-MS shows two major product peaks: reduced sCtLys¹⁸-Hynic and reduced sCtLys¹⁸-Hynic-TFA (in which the hydrazide is protected with a trifluoroacetyl group) identified by ES-MS. These two fractions are purified by reversed phase HPLC. The intramolecular disulfide bond is formed by aerial oxidation in 15 mL of helium-degassed 0.1M sodium bicarbonate pH8.2. RP-HPLC and ES-MS show that the non-protected hydrazine groups are vulnerable to N—N bond cleavage under these conditions, while the TFA protection prevent this.

The oxidised product sCtLys¹⁸-Hynic-TFA is purified by preparative RP HPLC, frozen in liquid nitrogen and then freeze-dried. Post-disulfide bond oxidation, analytical RP HPLC ES-MS of the target product peak is consistent with m/z (relative abundance) shown in the table below:

| | Observed | Calculated |
|---|---|---|
| Charged species | m/z | m/z |
| {C₁₅₃H₂₄₄F₃N₄₇O₅₀S₂ Average MW 3663.1} | | |
| [sCtLys¹⁸-Hynic-TFA+2H]²⁺ | 1832.5 (100) | 1832.6 |
| [sCtLys¹⁸-Hynic-TFA+3H]³⁺ | 1221.9 (34) | 1222.0 |

### Example 3

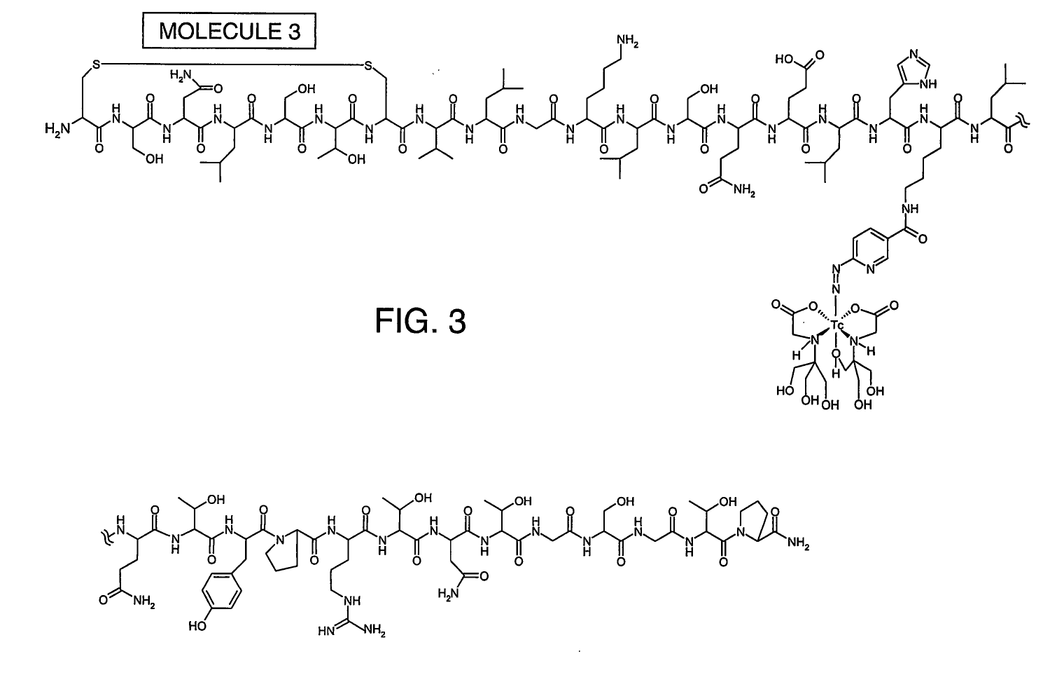

Synthesis of hynic-modified salmon calcitonin labelled with technetium-99m/tricine (Molecule 3, FIG. 3), and its affinity for human calcitonin receptors. Prior to radiolabelling, the TFA protecting group is removed, as follows: To 100 μL of sCtLys¹⁸-Hynic-TFA (0.275 mg/mL) in 10 mM sodium bicarbonate pH8.2, is added 200 μL of 0.1% TFA in H₂O. The mixture is left at room temperature for 6.75 h, pH2.5. A 21.8 μL aliquot containing 2.0 μg of peptide is treated with 50 μL of tricine (100 mg/mL in H₂O pH3.5), 5 μL of SnCl_{2 (}0.2 mg/mL in H₂O) and 100 μL of Na^{99m}TcO₄ (250 MBq) in physiological saline. The mixture is left at room temperature for 60 minutes. Analysis of the product by radiochromatography (instant thin layer chromatography and reversed phase HPLC) shows that 92-96% or the radioactivity is eluted in a single peak corresponding to labelled peptide, with the remainder eluting as pertechnetate. After incubation in human serum for 30 min, reversed phase HPLC and size exclusion chromatography show that the product is stable with no significant pertechnetate formation or binding of radioactivity to serum proteins. A triplicate calcitonin receptor binding assay with MCF-7 human breast cancer cells shows strong, specific receptor binding of the radiolabelled: at a concentration of 1 nM in a multi-well plate, the labelled peptide gives 23638 (SEM 490) counts per minute bound to MCF7 cells, 941 counts per minute bound to the well in the absence of cells, 726 counts per minute bound to cells in the presence of 1 μM cold salmon calcitonin, and 588 counts per minute bound to wells in the absence of cells but in the presence of 1 μm cold salmon calcitonin.

It will be understood that the invention has been described by way of example only and modifications may be made whilst remaining within the scope and spirit of the invention.

1. A chelator-derivatised amino acid comprising: 1) an optionally protected primary or secondary amino group; 2) a carboxylic acid group; 3) a chelator group capable of binding a metallic radionuclide.

2. A chelator-derivatised amino acid according to claim 1, wherein the chelator group is a hynic (hydrazinonicotinamide) group.

3. A chelator-derivatised amino acid according to claim 2 wherein the hynic hydrazine group is protected.

4. A chelator-derivatised amino acid according to claim 3 wherein the hynic hydrazine group is protected by a Boc or trifluoroacetyl protecting group.

5. A chelator-derivatised amino acid according to claim 1, wherein the amine and carboxylic acid groups are embodied in amino acid L-lysine or a homologue thereof.

6. A chelator-derivatised amino acid according to claim 5, wherein the amino acid is L-lysine or L-ornithine.

7. A chelator-derivatised amino acid according to claim 6, wherein the amino acid is L-lysine.

8. A chelator-derivatised amino acid according to claim 1, wherein the amino group is protected.

9. A chelator-derivatised amino acid according to claim 8, wherein the amino group is protected with an Fmoc protecting group.

10. A method of synthesising a peptide comprising a chelator, the method comprising incorporation into the peptide of a chelator-derivatised amino acid comprising: 1) an optionally protected primary or secondary amino group; 2) a carboxylic acid group; 3) a chelator group capable of binding a metallic radionuclide.

11. A method according to claim 10 wherein the peptide is synthesized by solid phase peptide synthesis.

12. A peptide obtained according to a method of claims 10.

13. A peptide comprising the amino acid sequence of salmon calcitonin in which lysine-18 is replaced by lysine-hynic.
